(19) United States
(12) Patent Application Publication
(10) Pub. No.: US 2020/0079710 A1
(43) Pub. Date: Mar. 12, 2020

(54) A METHOD FOR THE PRODUCTION OF HIGH PURITY BUTADIENE AND N-BUTENE FROM N-BUTANE USING AN OXIDATIVE DEHYDROGENATION PROCESS IN A CONTINUOUS-FLOW MULTI-LAYER-CATALYST FIXED-BED REACTOR

(71) Applicant: SABIC Global Technologies B.V., Bergen op Zoom (NL)

(72) Inventors: Tarek Jamal JAMALEDDINE, Riyadh (SA); YongMan CHOI, Riyadh (SA); Ramsey BUNAMA, Riyadh (SA)

(21) Appl. No.: 16/462,686

(22) PCT Filed: Dec. 4, 2017
(86) PCT No.: PCT/IB2017/057619

§ 371 Date: May 21, 2019

Related U.S. Application Data
(60) Provisional application No. 62/431,220, filed on Dec. 7, 2016.

(51) Int. Cl. C07C 5/48; C07C 5/333; B01J 8/04; B01J 19/00

(52) U.S. Cl. CPC C07C 5/48; C07C 11/167; B01J 8/0484; B01J 19/0046; B01J 2208/00814; B01J 2219/00033; B01J 2208/00884; B01J 2208/025; C07C 2521/06; C07C 2521/10; C07C 2523/06; C07C 2523/745; C07C 2523/755; C07C 2523/80; C07C 2523/847; C07C 5/3335

(57) ABSTRACT

Systems and methods for the production of n-butene isomers and/or 1,3-butadiene are disclosed. The systems and method involve an oxidative dehydrogenation (ODH) process for the production of n-butene isomers and 1,3-butadiene light olefins using an adjustable, multi-purpose, and multi-layer-catalyst bed for a reactor.

### CROSS REFERENCE TO RELATED APPLICATIONS

This application claims the benefit of priority of U.S. Provisional Patent Application No. 62/431,220, filed Dec. 7, 2016, which is hereby incorporated by reference in its entirety.

### FIELD OF INVENTION

The present invention generally relates to the production of light olefins. More specifically, the present invention relates to the oxidative dehydrogenation of C₄ hydrocarbon feedstock in a reactor that includes an adaptable multi-layer catalyst bed.

### BACKGROUND OF THE INVENTION

Market demand for the production of n-butene (CH₃CH₂CH═CH₂) and 1,3-butadiene (H₂C═CH—CH═CH₂) is gradually increasing. Both n-butene and 1,3-butadiene are used as raw material for various synthetic rubber and copolymer products. Conventionally, n-butene and 1,3-butadiene are produced from a naphtha cracking process; but this process is not dedicated to the production of these products. In other words, n-butene and 1,3-butadiene are by-products, and not the primary focus, of the naptha cracking process.

Due to the increased demand for n-butene and 1,3-butadiene, new facilities and/or expansion of naphtha cracking plants may be needed for increasing the production of n-butene and 1,3-butadiene. One process for the production of 1,3-butadiene that has been tried and has failed commercially is the direct dehydrogenation process. The direct dehydrogenation process has been shown to be inadequate as a suitable commercial process for the production of 1,3-butadiene from n-butene feed because the reaction for this process is very endothermic; thus, a large amount of energy is required to sustain the reaction and to burn-off unreacted carbon deposit on the surface of catalyst used in this process.

In response to the above challenges, an oxidative dehydrogenation (ODH) process has been gaining momentum in recent years as an effective alternative to produce n-butene and 1,3-butadiene from a C₄ mixture primarily containing n-butane reactant and including n-butene isomers (1-butene and 2-butene).

The following publications describe methods for the conversion of n-butene to produce 1,3-butadiene with high yield: U.S. Pat. No. 8,222,472 entitled “Method Of Producing 1,3-Butadiene From N-Butene Using Continuous-Flow Dual-Bed Reactor,” US Publication No. 2013/0090509 entitled “Single-Step Precipitation Method Of Producing Magnesia-Zirconia Complex Carrier For Catalyst For Oxidative Dehydrogenation Of N-Butane, Magnesium Orthovanadate Catalyst Supported On Magnesia-Zirconia Complex Carrier, And Method Of Producing N-Butene And 1,3-Butadiene Using Said Catalyst,” and US Publication No. US2011/0245568 entitled “Dehydrogenation Reactions Of N-Butene To Butadiene.” However, methods described in these publications have many side reactions that generate carbon oxides, namely carbon monoxide (CO) and carbon dioxide (CO₂), which is a drawback for these systems because the generation of carbon oxides to the atmosphere causes the greenhouse effect. The above mentioned publications also describe methods for the conversion of n-butane to produce 1,3-butadiene with high yield using multiple separate reactor systems.

### BRIEF SUMMARY OF THE INVENTION

A discovery has been made of systems and methods for the production of n-butene isomers and 1,3-butadiene that avoid the foregoing problems. In embodiments, the discovered systems and methods implement an oxidative dehydrogenation (ODH) process for the production of n-butene isomers and/or 1,3-butadiene light olefins using an adjustable, multi-purpose, and multi-layer-catalyst bed in a reactor. The different layers of catalyst bed may be separated by layers of non-reactive material. According to embodiments of the invention, a high purity n-butane gas feed (99 wt. %) may be co-fed with O₂ and steam into an ODH reactor equipped with a multi-layer catalyst-bed system to convert it to high purity 1,3-butadiene, or n-butene, or 1,3-butadiene and n-butent.

Embodiments of the invention include a method of producing n-butene (CH₃CH₂CH═CH₂) and/or 1,3-butadiene (H₂C═CH—CH═CH₂). The method may include flowing a feed stream comprising C₄ hydrocarbons, including n-butane (C₄H₁₀), to a reactor. The reactor may include a catalyst bed that comprises three separate catalytic layers arranged in series with respect to the flow of the feed stream. A first inert layer of material may be disposed between a first catalytic layer of the three separate catalytic layers and a second catalytic layer of the three separate catalytic layers. A second inert layer of material may be disposed between the second catalytic layer and a third catalytic layer of the three separate catalytic layers. The method may further include contacting the n-butane with the first catalytic layer under reaction conditions sufficient to convert n-butane to n-butene and 1,3-butadiene. The first catalytic layer may be adapted to catalyze the conversion of n-butane to n-butene and 1,3-butadiene. The method may further include flowing n-butene and/or 1,3-butadiene from the reactor.

Embodiments of the invention include a method of producing n-butene (CH₃CH₂CH═CH₂) and/or 1,3-butadiene (H₂C═CH—CH═CH₂). The method may include flowing a feed stream comprising C₄ hydrocarbons, including n-butane (C₄H₁₀), to a reactor. The reactor may include a catalyst bed that comprises three separate catalytic layers arranged in series with respect to the flow of the feed stream. A first inert layer of material may be disposed between a first catalytic layer of the three separate catalytic layers and a second catalytic layer of the three separate catalytic layers. A second inert layer of material may be disposed between the second catalytic layer and a third catalytic layer of the three separate catalytic layers. The method may further include contacting the n-butane with the first catalytic layer under reaction conditions sufficient to convert n-butane to n-butene and 1,3-butadiene. The first catalytic layer may be adapted to catalyze the conversion of n-butane to n-butene and 1,3-butadiene. The method may further include contacting a first portion of the n-butene with the second catalytic layer under reaction conditions sufficient to convert the first portion of the n-butene to 1,3-butadiene. The second catalytic layer may be adapted to catalyze conversion of n-butene to 1,3-butadiene. The method may further include contacting a second portion of the n-butene with the third catalytic layer under reaction conditions sufficient to convert the second portion of the n-butene to 1,3-butadiene, wherein the third catalytic layer is adapted to catalyze conversion of n-butene to 1,3-butadiene. The method may further include flowing n-butene and/or 1,3-butadiene from the reactor.

Embodiments of the invention include an apparatus for catalyzing reactions. The apparatus may include a multi-layer catalyst bed that comprises a first catalytic layer and a second catalytic layer, where a first inert layer is disposed between the first catalytic layer and the second catalytic layer. The apparatus may further include a third catalytic layer and a second inert layer disposed between the second catalytic layer and the third catalytic layer. The catalytic layers may be adapted to receive flow of reactant gases, where the catalytic layers and inert layers are arranged in series with respect to the flow of the reactant gases.

The following includes definitions of various terms and phrases used throughout this specification.

The terms “about” or “approximately” are defined as being close to as understood by one of ordinary skill in the art. In one non-limiting embodiment the terms are defined to be within 10%, preferably, within 5%, more preferably, within 1%, and most preferably, within 0.5%.

The terms “wt. %”, “vol. %” or “mol. %” refers to a weight, volume, or molar percentage of a component, respectively, based on the total weight, the total volume, or the total moles of material that includes the component. In a non-limiting example, 10 moles of component in 100 moles of the material is 10 mol. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification, includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with the term “comprising,” “including,” “containing,” or “having” in the claims or the specification may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The process of the present invention can “comprise,” “consist essentially of,” or “consist of” particular ingredients, components, compositions, etc., disclosed throughout the specification.

The term “primarily” as that term is used in the specification and/or claims, means greater than 50%, e.g., 50 wt. %, 50 mol. %, and/or 50 vol. %, etc., for example, from 50.01 to 100.00%, preferably 51% to 99%, and more preferably 60% to 90%.

In the context of the present invention, twenty embodiments are now described. Embodiment 1 is a method of producing n-butene (CH₃CH₂CH═CH₂) and/or 1,3-butadiene (H₂C═CH—CH═CH₂), the method including the steps of flowing a feed stream containing C₄ hydrocarbons, including n-butane (C₄H₁₀), to a reactor, the reactor including a catalyst bed that includes three separate catalytic layers arranged in series with respect to the flow of the feed stream, wherein a first inert layer of material is disposed between a first catalytic layer of the three separate catalytic layers and a second catalytic layer of the three separate catalytic layers, wherein a second inert layer of material is disposed between the second catalytic layer and a third catalytic layer of the three separate catalytic layers, contacting the n-butane with the first catalytic layer under reaction conditions sufficient to convert n-butane to n-butene and 1,3-butadiene, wherein the first catalytic layer is adapted to catalyze conversion of n-butane to n-butene and 1,3-butadiene; and flowing n-butene and/or 1,3-butadiene from the reactor. Embodiment 2 is the method of embodiment 1, wherein the feed stream contains primarily n-butane. Embodiment 3 is the method of any of embodiments 1 and 2, wherein the feed stream contains 85 to 99 wt. % n-butane, 1 to 10 wt. % of n-butene, and 0 to 5 wt. % of residual C₄ compounds. Embodiment 4 is the method of any of embodiments 1 to 3, wherein each catalytic layer contains different catalytic materials from the other catalytic layers. Embodiment 5 is the method of any of embodiments 1 to 4, further including the step of contacting a first portion of the n-butene with the second catalytic layer under reaction conditions sufficient to convert the first portion of the n-butene to 1,3-butadiene, wherein the second catalytic layer is adapted to catalyze conversion of n-butene to 1,3-butadiene. Embodiment 6 is the method of embodiment 5, further including the step of contacting a second portion of the n-butene with the third catalytic layer under reaction conditions sufficient to convert the second portion of the n-butene to 1,3-butadiene, wherein the third catalytic layer is adapted to catalyze conversion of n-butene to 1,3-butadiene. Embodiment 7 is the method of any of embodiments 1 to 6, wherein the first catalytic layer contains magnesium orthovanadate (O-Vanadate) catalyst (Mg₃(VO₄)₂) supported by a magnesia-zirconia complex. Embodiment 8 is the method of any of embodiments 1 to 7, wherein the second catalytic layer contains zinc ferrite catalyst. Embodiment 9 is the method of any of embodiments 1 to 8, wherein the third catalytic layer contains bismuth molybdate catalyst. Embodiment 10 is the method of any of embodiments 1 to 9, further including the step of separating a stream containing 1,3-butadiene and n-butane, with or without 1-butene and 2-butene, into a steam containing n-butane, with or without 1-butene and 2-butene, and a stream containing 1,3-butadiene. Embodiment 11 is the method of embodiment 10, further including the step of recycling the stream containing n-butane, with or without 1-butene and 2-butene as feed. Embodiment 12 is the method of any of embodiments 1 to 11, wherein the feed stream includes air and a ratio of n-butane:air is 10:40 to 10:50 by volume. Embodiment 13 is the method of any of embodiments 1 to 12, wherein an oxidative dehydrogenation reaction at the first catalytic layer is conducted at a reaction temperature of 500° C. to 600° C. and a gas hourly space velocity (GHSV) of 300 h-1 to 600 h-1. Embodiment 14 is the method of any of embodiments 1 to 13, wherein the first catalytic layer includes iron, nickel, titanium, vanadium, and magnesium. Embodiment 15 is the method of any of embodiments 1 to 14, wherein the third catalytic layer may include iron and a selection from the list consisting of: potassium, magnesium, zirconium, chromium, nickel, cobalt, tin, lead, germanium, manganese, silicon, aluminum, chromium, tungsten, phosphorous, and lanthanum, or combinations thereof. Embodiment 16 is the method of any of embodiments 1 to 15, further including the step of removing catalyst in the second catalytic layer and the third catalytic layer and replacing the removed catalyst from the second catalytic layer and the third catalytic layer with magnesium orthovanadate (O-Vanadate) catalyst. Embodiment 17 is the method of any of embodiments 1 to 16, wherein the selectivity for n-butene is at least 98% to 99% and the method further includes the steps of isomerizing the n-butene to isobutylene; and introducing the isobutylene into a mixing reactor with methanol to form MTBE.

Embodiment 18 is an apparatus for catalyzing reactions. The apparatus includes a multi-layer catalyst bed including a first catalytic layer; a second catalyst layer; a first inert layer disposed between the first catalytic layer and the second catalytic layer: a third catalytic layer; a second inert layer disposed between the second catalytic layer and the third catalytic layer, wherein the catalytic layers are adapted to receive flow of reactant gases, wherein the catalytic layers and inert layers are arranged in series with respect to the flow of the reactant gases. Embodiment 19 is the apparatus of embodiment 18, wherein the apparatus is adapted so that catalyst used in any of the first catalytic layer, second catalytic layer, or third catalytic layer is replaceable without having to replace the catalyst of the other catalytic layers. Embodiment 20 is the apparatus of any of embodiments 18 and 19, wherein catalyst in the first catalytic layer, catalyst in the second catalytic layer, and catalyst in the third catalytic layer are different from each other and the apparatus further includes a frame for receiving and supporting a plurality of trays, each of the trays containing at least one of the catalytic layers, wherein each of the trays is removable from the frame without removing the other trays.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description. In further embodiments, features from specific embodiments may be combined with features from other embodiments. For example, features from one embodiment may be combined with features from any of the other embodiments. In further embodiments, additional features may be added to the specific embodiments described herein.

### BRIEF DESCRIPTION OF THE DRAWINGS

For a more complete understanding, reference is now made to the following descriptions taken in conjunction with the accompanying drawings, in which:

FIG. 1 shows a schematic of a reactor system for the production of n-butene and/or 1,3-butadiene, according to embodiments of the invention;

FIG. 2 shows a catalyst bed, according to embodiments of the invention;

FIG. 3 shows a catalyst bed, according to embodiments of the invention;

FIG. 4 shows a tray for holding catalyst in a catalyst bed, according to embodiments of the invention;

FIG. 5 shows a tray for holding catalyst in a catalyst bed, according to embodiments of the invention; and

FIG. 6 shows a flow diagram for the production of n-butene and/or 1,3-butadiene, according to embodiments of the invention.

### DETAILED DESCRIPTION OF THE INVENTION

A discovery has been made of systems and methods for the production of n-butene isomers and/or 1,3-butadiene that avoid the problems discussed above with conventional systems for producing n-butene and/or 1,3-butadiene. In embodiments, the discovered systems and methods implement an oxidative dehydrogenation (ODH) process for the production of n-butene isomers and 1,3-butadiene light olefins using an adjustable, multi-purpose, and multi-layer-catalyst bed for a reactor. The different layers of the catalyst bed may be separated physically by disposing a layer of inert or powder-like material between them (buffer) that has no reactivity when exposed to the materials (reactants and products) under the conditions in the reactor. For example, the layer of inert material is stable at high temperatures that occur in the reactor (a non-reactive layer).

Implementing the ODH process with the adjustable, multi-purpose, and multi-layer-catalyst bed, according to embodiments of the invention, result in high yield of n-butene and/or 1,3-butadiene, while producing less carbon oxides (CO and CO₂) than conventional processes. Further, the adjustability of the multi-functional aspects of the catalyst bed provides an economical method for varying the concentration and selectivity of either n-butene or 1,3-butadiene, depending on, for example, the market demand for each of these products. In other words, depending on whether n-butene or 1,3-butadiene is in higher demand than the other, or whether they are equally in demand, the process may be economically adjusted to produce (1) only n-butene or primarily n-butene; (2) only 1,3-butadiene or primarily 1,3-butadiene; or (3) n-butene and 1,3-butadiene equally or substantially equally.

According to embodiments of the invention, instead of major changes in infrastructure and/or modification to include additional components to reactor systems to meet market demand, existing reactors may be retrofitted with the adjustable, multi-purpose, and multi-layer reactor beds described herein. With such adjustable, multi-purpose, and multi-layer reactor beds, adjusting the production process to meet market demand for n-butene or 1,3-butadiene is more economical than the major redesigns and additions that would have to be made to conventional systems. According to embodiments of the invention, the catalyst used in each of the layers of the multi-layer catalyst bed may be changed without changing the catalyst in another layer. Modifying the catalyst makeup of the catalyst bed in this way can vary the production of n-butene isomers in relation to 1,3-butadiene, according to market demand.

In embodiments of the invention, the ODH process is implemented to produce n-butene isomers and 1,3-butadiene from a C₄ hydrocarbon mixture of primarily n-butane in a continuous flow single reactor system. In embodiments of the invention, the C₄ hydrocarbon mixture supplied to the ODH process used to produce n-butene isomers and 1,3-butadiene is a high purity n-butane feed.

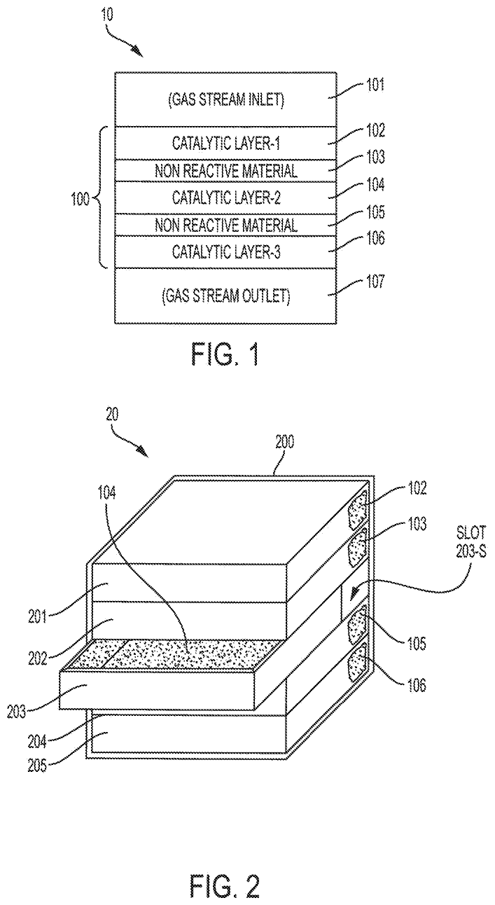

FIG. 1 shows a schematic of continuous flow single reactor system 10 for the production of n-butene and/or 1,3-butadiene, according to embodiments of the invention. As illustrated in FIG. 1, reactor system 10 includes catalyst bed 100. FIG. 1 shows reactor system 10 in a vertical orientation; however, in embodiments of the invention, reactor system 10 may be oriented differently, e.g., reactor system 10 may be oriented horizontally. In embodiments of the invention, reactor inlet 101 leads to catalyst bed 100. Catalyst bed 100 may include a plurality of layers of catalytic material as well non-catalytic/non-reactive (inert) material arranged in series with respect to the flow of reactant gases through reactor system 10. The flow of reactor gases further to embodiments of the invention, includes flow through reactor inlet 101 to catalytic layer 102, from catalytic layer 102 to non-reactive layer 103, from non-reactive layer 103 to catalytic layer 104, from catalytic layer 104 to non-reactive layer 105, from non-reactive layer 105 to catalytic layer 106, and from catalytic layer 106 through reactor outlet 107.

FIG. 1 shows that, in embodiments of the invention, catalyst bed 100 may be configured so that reactor inlet 101 leads to catalytic layer 102, which may be disposed adjacent to non-reactive layer 103. And non-reactive layer 103 may be disposed adjacent catalytic layer 104. Further, catalytic layer 104 may be disposed adjacent non-reactive layer 105 and non-reactive layer 105 may be disposed adjacent catalytic layer 106. Reactor outlet 107 may lead from catalytic layer 106. Catalytic layer 102, catalytic layer 104, and catalytic layer 106 may include different catalysts. However, in embodiments of the invention one or more of catalytic layer 102, catalytic layer 104, and catalytic layer 106 may include the same catalyst material.

In embodiments of the invention, the layers that are adjacent each other may be in contact with each other. For example, one side of catalytic layer 102 may be in contact with a first side of non-reactive layer 103. In turn, the second side of non-reactive layer 103 may be in contact with a first side of catalytic layer 104. A second side of catalytic layer 104 may be in contact with a first side of non-reactive layer 105.

Alternatively or additionally, in embodiments of the invention, the layers that are adjacent each other may not be in physical contact with each other. For example, catalytic layer 102 may be disposed in a tray having a base with holes of sufficient size so that reactant gases will flow through the holes but particles of catalytic layer 102 will not. In this way, the tray provides support for catalytic layer 102 while separating catalytic layer 102 from direct contact with non-reactive layer 103, even though catalytic layer 102 and non-reactive layer 103 are close to each other. One or more of the layers may be supported by a tray which separates the one or more layers from other layers. In embodiments of the invention, any of catalytic layers 102, 104, and 106; non-reactive layers 103 and 105; or combinations thereof, may be supported or not supported by a tray.

For example, each of the layers shown in FIG. 1, namely catalytic layer 102, non-reactive layer 103, catalytic layer, 104, non-reactive layer 105, and catalytic layer 106 may each have trays that carry and support them, where the base of each tray separates the layer it is supporting from the layer adjacent to the layer being supported.

FIG. 2 shows catalyst bed 20, according to embodiments of the invention that may be used to implement reactor system 10 shown in FIG. 1. Catalyst bed 20 may include frame 200 for receiving and supporting trays 201 to 205 into slots within frame 200 (e.g., slot 203-S is adapted to receive tray 203, which is shown in FIG. 2 being partially outside of frame 200). According to embodiments of the invention, catalyst material that makes up catalytic layer 102 may be placed in tray 201. Tray 201 includes openings (e.g., holes) in its base that are big enough to allow reactant gases to flow from catalytic layer 102 to non-reactive layer 103; but the openings are small enough so that the particles of catalytic layer 102 do not go through the openings. In this way, according to embodiments of the invention, catalytic layer 102 is separated from non-reactive layer 103 by at least the thickness of the bottom portion of tray 201, e.g., the thickness of a perforated metal plate that forms the base of tray 201. Similarly, in embodiments of the invention, tray 202 supports non-reactive layer 103 and separates non-reactive layer 103 from catalytic layer 104, tray 203 supports catalytic layer 104 and separates catalytic layer 104 from non-reactive layer 105, tray 204 supports non-reactive layer 105 and separates non-reactive layer 105 from catalytic layer 106; and tray 205 supports catalytic layer 106. FIG. 2 includes “broken-out” sections of trays 201 to 205 to show the respective layers disposed in trays 201 to 205.

As a further example of trays providing support for one or more layers, catalytic layer 102 may be in direct contact with (by resting on top of) non-reactive layer 103, where both catalytic layer 102 and non-reactive layer 103 are supported by a first tray below and in contact with non-reactive layer 103. Similarly, catalytic layer 104 may be in direct contact with non-reactive layer 105, where both catalytic layer 104 and non-reactive layer 105 are supported by a second tray below non-reactive layer 105. A third tray may support catalytic layer 106.

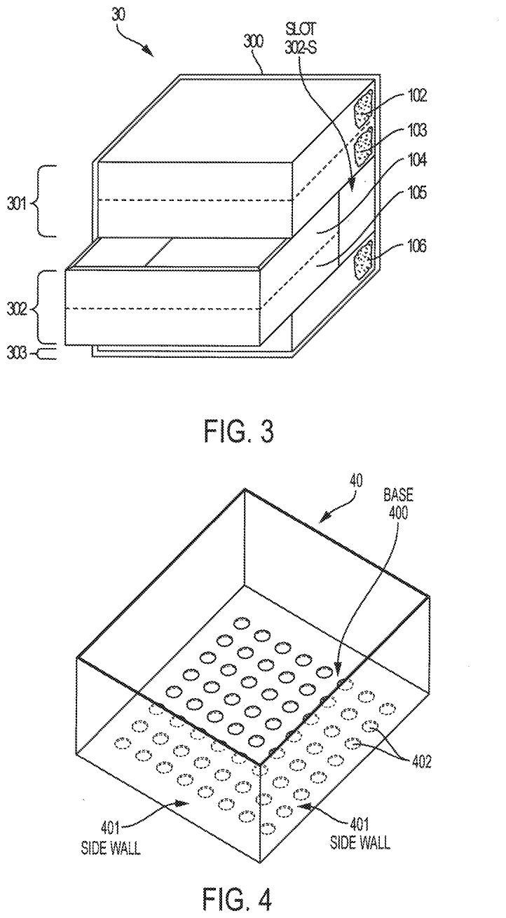

FIG. 3 shows a catalyst bed, according to embodiments of the invention, illustrating the example of a tray supporting more than one layers of the catalyst bed. Catalyst bed 30 may include frame 300 for receiving trays 301 to 303 in slots within frame 300 (e.g., slot 302-S for tray 302). According to embodiments of the invention, catalytic layer 102 may be in direct contact with (e.g., directly on top of) non-reactive layer 103, which are both placed in and supported by tray 301. Tray 301, according to embodiments of the invention, includes openings (e.g., holes) in its base that are big enough to allow reactant gases to flow from catalytic layer 102 and non-reactive layer 103 to catalytic layer 104; but the openings are small enough so that the particles of non-reactive layer 103 do not go through the openings. In this way, according to embodiments of the invention, catalytic layer 102 and non-reactive layer 103 are separated from catalytic layer 104 by at least the thickness of the bottom portion of tray 301, e.g., the thickness of a perforated metal plate that forms the base of tray 301. Similarly, in embodiments of the invention, catalytic layer 104 may be in direct contact with (e.g., directly on top of) non-reactive layer 105, which are both placed in and supported by tray 302. In this way, catalytic layer 104 and non-reactive layer 105 are separated from catalytic layer 106 by at least the thickness of the bottom portion of tray 302. Catalytic layer 106 may be held in and supported by tray 303. FIG. 3 includes “broken-out” sections of trays 301 to 303 to show the respective layers disposed in trays 301 to 303.

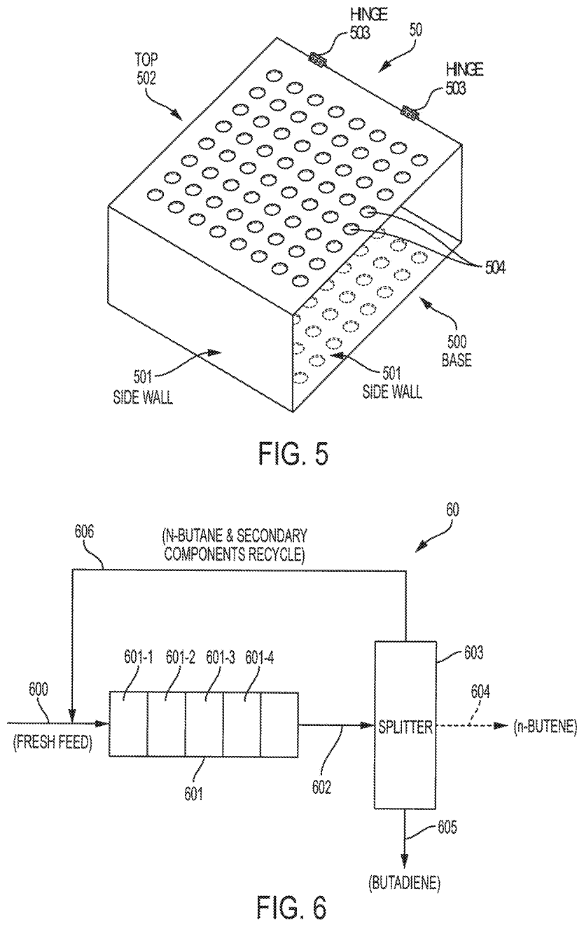

In embodiments of the invention, non-reactive materials between catalytic layers may include non-reactive layers 103 and 105 and/or trays 201 to 205 and trays 301 to 303. In embodiments of the invention, trays 201 to 205 and trays 301 to 303 may or may not include a top with openings similar to the base with openings. For example, FIG. 4 shows tray 40 having base 400 (with holes 402), side walls 401, and no top. FIG. 5 shows tray 50 having base 500 (with holes 504), side walls 501, top 502 (with holes 504), and hinges 503. Hinges 503 may allow for top 502 to be temporarily moved so that the catalytic material in tray 50 can be removed and replaced. The trays described herein may be made of materials that can withstand being exposed to reactants and products in the reactor and the conditions in the reactor. In embodiments of the invention, the trays may be made of similar or same material of which the reactor is made. It should be noted that the use of trays as described herein is just one example of implementing the separation of catalytic layers and/or non-reactive layers in a multi-layer catalyst bed and providing a way to easily modify the catalyst used in each layer. Accordingly, the separation of layers and easily modified functionalities of the catalyst bed, in embodiments of the invention, may be implemented by alternative or additional systems.

Further to the systems and apparatus of FIG. 1 to FIG. 5, embodiments of the invention may include an apparatus for catalyzing reactions. The apparatus may include a multi-layer catalyst bed that comprises a first catalytic layer and a second catalyst layer. The apparatus may also include a first inert layer disposed between the first catalytic layer and the second catalytic layer. The apparatus may further include a third catalytic layer and a second inert layer disposed between the second catalytic layer and the third catalytic layer. The catalytic layers are adapted to receive flow of reactant gases and the catalytic layers and inert layers may be arranged in series with respect to the flow of the reactant gases. In embodiments of the invention, the catalyst in the first catalytic layer, catalyst in the second catalytic layer, and catalyst in the third catalytic layer are different from each other. However, in view of the adaptability of the reactor beds described herein, in embodiments of the invention, one or more of the catalytic layers may be adapted to include the same catalyst material.

FIG. 6 shows flow diagram 60 for the production of n-butene and/or 1,3-butadiene, according to embodiments of the invention. The process of producing n-butene and/or 1,3-butadiene may begin, as shown in flow diagram 60, by flowing fresh feed 600 to catalytic dehydrogenation unit 601. In embodiments of the invention, fresh feed 600 comprises C₄ hydrocarbons, including n-butane (C₄H₁₀), oxygen, and steam. In embodiments of the invention, fresh feed 600 may comprise primarily n-butane. Further, in embodiments of the invention, fresh feed 600 may comprise 85 to 99 wt. % n-butane, 1 to 10 wt. % of n-butene, and 0 to 5 wt. % of residual C₄ compounds. Further yet, in embodiments of the invention, fresh feed 600 may include air and a ratio of n-butane:air:steam is approximately 10:40:50 by volume.

Fresh feed 600 may be fed into dehydrogenation zone 601-1, which is a first catalytic layer that may comprise magnesium orthovanadate (O-Vanadate) catalyst supported by a magnesia-zirconia complex carrier. In embodiments of the invention, at dehydrogenation zone 601-1, the oxidative dehydrogenation reaction is conducted at a reaction temperature of 500 to 600° C. and a gas hourly space velocity (GHSV) of 300 to 600 h⁻¹. According to embodiments of the invention, in dehydrogenation zone 601-1, the oxidative dehydrogenating of n-butane to 1-butene, 2-butene, 1,3-butadiene and water occurs, which results in a first product stream comprising unconverted n-butane, n-butene, 1,3-butadiene, and secondary components. Catalysts that are particularly suitable for the oxydehydrogenation of n-butane to n-butenes and 1,3-butadiene include those generally based on supported vanadium catalyst such as orthovanadate (O-Vanadate) catalyst which generally includes iron, nickel, titanium, vanadium, and magnesium.

Conversion of fresh feed 600, when it contacts magnesium orthovanadate (O-Vanadate) catalyst (Mg₃(VO₄)₂) supported by a magnesia-zirconia complex carrier, at a temperature of 500° C. to 600° C., to a mixture containing primarily n-butene & 1,3-butadiene may be at a rate in the order of 35 wt. % and the selectivity of products may be approximately 52 wt. %.

In embodiments of the invention, the first product gas stream, which may comprise unconverted n-butane, 1-butene, 2-butene, 1,3-butadiene and secondary components, is flowed into dehydrogenation zone 601-2, which may comprise zinc ferrite catalyst as a second catalyst layer to catalyze reactants to produce a second product stream. The layer of zinc ferrite catalyst favors the conversion of n-butene to 1,3-butadiene with conversion and selectivity of 78 wt. % and 92 wt. %, respectively. In this way, the process may include contacting a first portion of the n-butene with the second catalytic layer under reaction conditions sufficient to convert the first portion of the n-butene to 1,3-butadiene, where the second catalytic layer is adapted to catalyze conversion of n-butene to 1,3-butadiene.

For obtaining even additional conversion of unconverted n-butane and n-butene fractions and to obtain higher 1,3-butadiene selectivity, the second product stream may then be contacted with a layer of multicomponent bismuth molybdate catalyst to convert it to a high purity 1,3-butadiene with selectivity and yield rates of 97 wt. % and 82 wt. %, respectively. Considering this in view of FIG. 6, non-reactive layer 601-3 may be disposed between dehydrogenation zone 601-2 and dehydrogenation zone 601-4. Dehydrogenation zone 601-4 may comprise bismuth molybdate-based as a third catalyst layer. In this way, the process may include contacting a second portion of the n-butene with the third catalytic layer under reaction conditions sufficient to convert the second portion of the n-butene to 1,3-butadiene, wherein the third catalytic layer is adapted to catalyze conversion of n-butene to 1,3-butadiene. It should be noted that catalysts which are particularly suitable for the oxydehydrogenation of the n-butenes to 1,3-butadiene, and which may be used in the third catalyst layer, are generally based on an Mo—Bi—O multi-metal oxide system which generally comprises iron and additional components such as potassium, magnesium, zirconium, chromium, nickel, cobalt, tin, lead, germanium, manganese, silicon, aluminum, chromium, tungsten, phosphorous, or lanthanum.

The catalyst layers of dehydrogenation zone 601-2 and 601-4 causes the oxidative dehydrogenating of 1-butene and 2-butene from the first product stream to obtain product gas stream 602, which may comprise primarily 1,3-butadiene and secondary components. Splitter 603 may separate product gas stream 602 (which may comprise 1,3-butadiene and unconverted n-butane, with or without 1-butene and 2-butene) into at least stream 604 (comprising N-butene), stream 605 (comprising 1,3 butadiene), and stream 606 (comprising n-butane and secondary components). Stream 606 may comprise n-butane, with or without 1-butene and 2-butene. Stream 606 may comprise n-butane, with or without 1-butene and 2-butene. In embodiments of the invention, stream 606 is recycled into dehydrogenation zone 601-1 as feed.

In embodiments of the invention, if the market demand for n-butene isomers is higher than the demand for 1,3-butadiene, 1-butene for synthetic rubber application or isobutylene for methyl tert butyl ether (MTBE) production, the production of high purity 1,3-butadiene can be substituted with the production of high purity 1-butene in the second and third catalyst layers, in dehydrogenation zone 601-2 and dehydrogenation zone 601-4, respectively. To do this, zinc ferrite and multicomponent bismuth molybdate catalysts may be removed from dehydrogenation unit 601 and replaced by one or more layers of oxidative catalyst (e.g., magnesium orthovanadate (O-Vanadate) catalyst (Mg₃(VO₄)₂) supported by a magnesia-zirconia complex) to convert the stream comprising n-butene, 1,3-butadiene and unconverted n-butane portions generated downstream of the first catalyst layer (dehydrogenation zone 601-1) into 1-butene. This illustrates that, in embodiments of the invention, depending on product demand, it may be preferable that the different layers in the catalyst bed have the same catalyst material. The catalyst beds described herein provides the ability to easily change the catalyst bed configuration as product demand dictates.

In embodiments of the invention, when the selectivity for n-butene is 98% to 99%, or higher, the method may further include isomerizing the n-butene to isobutylene and introducing the isobutylene into a mixing reactor with methanol to form MTBE. The final product can be used as raw material for the production of synthetic rubber, linear low density polyethylene (LLDPE) or MTBE.

Further to FIG. 1 to FIG. 6, embodiments of the invention include an apparatus for catalyzing reactions. The apparatus may include a multi-layer catalyst bed that may include a first catalytic layer, a second catalyst layer, and a first inert layer disposed between the first catalytic layer and the second catalytic layer. The apparatus may further include a third catalytic layer, a second inert layer disposed between the second catalytic layer and the third catalytic layer. The catalytic layers may be adapted to receive flow of reactant gases, where the catalytic layers and inert layers are arranged in series with respect to the flow of the reactant gases. The apparatus may further include a frame for receiving and supporting a plurality of trays. Each of the trays may include at least one of the catalytic layers, where each of the trays may be removable from the frame without removing the other trays so that catalyst used in any of the first catalytic layer, second catalytic layer, or third catalytic layer is replaceable without having to replace the catalyst of the other catalytic layers. In embodiments of the invention, the catalyst in the first catalytic layer, catalyst in the second catalytic layer, and catalyst in the third catalytic layer are different from each other.

The ODH process described herein can save energy, reduce capital and operational cost, and lower environmental impact by reducing greenhouse gas emissions. Energy can be saved because of the addition of oxygen, which initiates dehydrogenation by abstracting hydrogen and combusting it to supply heat required for the endothermic reaction. Capital cost can be reduced by eliminating the need for a furnace. Operational cost can be reduced by eliminating the need for decoking shutdowns, because oxygen assists in regenerating the catalyst during the dehydrogenation process. Further, embodiments of the invention reduce the formation of greenhouse gases, while still yielding high product selectivity and high conversion of n-butene.

Although embodiments of the present application and their advantages have been described in detail, it should be understood that various changes, substitutions and alterations can be made herein without departing from the spirit and scope of the embodiments as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the above disclosure, processes, machines, manufacture, compositions of matter, means, methods, or steps, presently existing or later to be developed that perform substantially the same function or achieve substantially the same result as the corresponding embodiments described herein may be utilized. Accordingly, the appended claims are intended to include within their scope such processes, machines, manufacture, compositions of matter, means, methods, or steps.

1. A method of producing n-butene (CH₃CH₂CH═CH₂) and/or 1,3-butadiene (H₂C═CH—CH═CH₂), the method comprising:
flowing a feed stream comprising C₄ hydrocarbons, including n-butane (C₄H₁₀), to a reactor, the reactor including a catalyst bed that comprises three separate catalytic layers arranged in series with respect to the flow of the feed stream, wherein a first inert layer of material is disposed between a first catalytic layer of the three separate catalytic layers and a second catalytic layer of the three separate catalytic layers, wherein a second inert layer of material is disposed between the second catalytic layer and a third catalytic layer of the three separate catalytic layers,
contacting the n-butane with the first catalytic layer under reaction conditions sufficient to convert n-butane to n-butene and 1,3-butadiene, wherein the first catalytic layer is adapted to catalyze conversion of n-butane to n-butene and 1,3-butadiene; and
flowing n-butene and/or 1,3-butadiene from the reactor.

2. The method of claim 1, wherein the feed stream comprises primarily n-butane.

3. The method of claim 1, wherein the feed stream comprises 85 to 99 wt. % n-butane, 1 to 10 wt. % of n-butene, and 0 to 5 wt. % of residual C₄ compounds.

4. The method of claim 1, wherein each catalytic layer comprises different catalytic materials from the other catalytic layers.

5. The method of claim 1, further comprising:
contacting a first portion of the n-butene with the second catalytic layer under reaction conditions sufficient to convert the first portion of the n-butene to 1,3-butadiene, wherein the second catalytic layer is adapted to catalyze conversion of n-butene to 1,3-butadiene.

6. The method of claim 5, further comprising:
contacting a second portion of the n-butene with the third catalytic layer under reaction conditions sufficient to convert the second portion of the n-butene to 1,3-butadiene, wherein the third catalytic layer is adapted to catalyze conversion of n-butene to 1,3-butadiene.

7. The method of claim 1, wherein the first catalytic layer comprises magnesium orthovanadate (O-Vanadate) catalyst (Mg₃(VO₄)₂) supported by a magnesia-zirconia complex.

8. The method of claim 1, wherein the second catalytic layer comprises zinc ferrite catalyst.

9. The method of claim 1, wherein the third catalytic layer comprises bismuth molybdate catalyst.

10. The method of claim 1, further comprising:
separating a stream comprising 1,3-butadiene and n-butane, with or without 1-butene and 2-butene, into a steam comprising n-butane, with or without 1-butene and 2-butene, and a stream comprising 1,3-butadiene.

11. The method of claim 10, further comprising:
recycling the stream comprising n-butane, with or without 1-butene and 2-butene as feed.

12. The method of any of claim 1, wherein the feed stream includes air and a ratio of n-butane:air is 10:40 to 10:50 by volume.

13. The method of any of claim 1, wherein an oxidative dehydrogenation reaction at the first catalytic layer is conducted at a reaction temperature of 500° C. to 600° C. and a gas hourly space velocity (GHSV) of 300 h⁻¹ to 600 h⁻¹.

14. The method of any of claim 1, wherein the first catalytic layer includes iron, nickel, titanium, vanadium, and magnesium.

15. The method of any of claim 1, wherein the third catalytic layer may include iron and a selection from the list consisting of: potassium, magnesium, zirconium, chromium, nickel, cobalt, tin, lead, germanium, manganese, silicon, aluminum, chromium, tungsten, phosphorous, and lanthanum, or combinations thereof.

16. The method of any of claim 14, further comprising:
removing catalyst in the second catalytic layer and the third catalytic layer and replacing the removed catalyst from the second catalytic layer and the third catalytic layer with magnesium orthovanadate (O-Vanadate) catalyst.

17. The method of any of claim 1, wherein the selectivity for n-butene is at least 98% to 99% and the method further comprises:
isomerizing the n-butene to isobutylene; and
introducing the isobutylene into a mixing reactor with methanol to form MTBE.

18. An apparatus for catalyzing reactions, the apparatus comprising:
a multi-layer catalyst bed comprising:
a first catalytic layer;
a second catalyst layer;
a first inert layer disposed between the first catalytic layer and the second catalytic layer:
a third catalytic layer;
a second inert layer disposed between the second catalytic layer and the third catalytic layer, wherein the catalytic layers are adapted to receive flow of reactant gases, wherein the catalytic layers and inert layers are arranged in series with respect to the flow of the reactant gases.

19. The apparatus of claim 18, wherein the apparatus is adapted so that catalyst used in any of the first catalytic layer, second catalytic layer, or third catalytic layer is replaceable without having to replace the catalyst of the other catalytic layers.

20. The apparatus of claim 18, wherein catalyst in the first catalytic layer, catalyst in the second catalytic layer, and catalyst in the third catalytic layer are different from each other and the apparatus further comprises:
a frame for receiving and supporting a plurality of trays, each of the trays comprising at least one of the catalytic layers, wherein each of the trays is removable from the frame without removing the other trays.
